(19) United States
(12) Patent Application Publication
(10) Pub. No.: US 2010/0087776 A1
(43) Pub. Date: Apr. 8, 2010

(54) Process And Apparatus For Filling A Syringe With A Thoroughly Mixed Medical Gas Mixture

(75) Inventor: Tim J. Warren, Easton, PA (US)

Correspondence Address: AIR LIQUIDE;Intellectual Property, 2700 POST OAK BOULEVARD, SUITE 1800, HOUSTON, TX, 77056, US

(73) Assignee: Air Liquide Healthcare America Corporation, Houston, TX (US)

(21) Appl. No.: 12/568,328

(22) Filed: Sep. 28, 2009

Related U.S. Application Data
(60) Provisional application No. 61/102,480, filed on Oct. 3, 2008.

(51) Int. Cl. A61M 5/31; B65B 1/04

(52) U.S. Cl. 604/26; 141/2; 141/18

(57) ABSTRACT

The present invention provides a system for filling a syringe with a small amount of a more precisely mixed medical gas mixture, the system comprising two or more sources for gas, a device for mixing the gases, a means for connecting the gas sources to the device for mixing the gases, a filter system and a syringe. The present invention further provides for a process for filling a syringe with a small quantity of a thoroughly mixed medical gas mixture comprising at least a first gas and a second gas. The process comprises the steps of providing at least a first gas and a second gas, injecting the gases into a device for mixing to form a thoroughly mixed medical gas mixture, filtering the medical gas mixture, and filling a syringe with the required amount of medical gas mixture.

### CROSS-REFERENCE TO RELATED APPLICATIONS

This application claims the benefit of U.S. Provisional Application No. 61/102,480, filed Oct. 3, 2008, the entire contents of which are incorporated herein by reference.

### FIELD OF THE INVENTION

The present invention relates to a process for precisely mixing gases for medical use. More specifically, the present invention relates to a process for mixing medical gases and filling a syringe with small amounts of the resulting medical gas mixture for use in intraocular surgery. The present invention further relates to a system for accomplishing the same.

### BACKGROUND

Major strides have been made in ocular surgery over the past one hundred years. The use of medical gases in ocular surgery has also increased in major strides. The first use of a gas can be traced back to the early 1900's when air bubbles were used as intraocular tamponades of retinal detachment. Over the years, the use of different inert gases has become commonplace especially with regard to such gases as SF₆ and C₃F₈. In addition, many such medical gases have multiple established uses in ophthalmic procedures such as in pneumatic retinopexy, vitreous surgery and corneal surgery. In some of these procedures, pure gases are utilized. In other procedures such as vitreous surgery, pure gases are diluted mainly because of the ability of the gases utilized to expand in the human body. Because these gases have the ability to expand, it is very important that they be diluted and mixed properly in order to avoid injury to the eye that may be caused by increased intraocular pressure due to the expanding gases. Current practice is to draw a predetermined amount of gas into a syringe and then to draw the additional amount (the remainder) of additional gas into the syringe, perhaps followed by some means of agitation in order to mix the two gases. In some instances, the degree of mixing is insufficient to properly mix the two or more gases. As a result, in some instances this insufficient mixing can result in the injection into the eye of a gas mixture which is not a true representation of what the doctor believes is being injected. Damage could result from this insufficient mixing since the gas mixture injected could expand.

Accordingly, there exists a need to have a process that allows for more precise mixing than is currently observed with regard to the prior art processes. In addition, there exists a need for a system that will accomplish the same.

### SUMMARY OF THE INVENTION

The present invention provides a system for filling a syringe with a small amount of a more precisely mixed medical gas mixture, the system comprising two or more sources for gas, a device for mixing the gases, a means for connecting the gas sources to the device for mixing the gases, a filter system and a syringe. The present invention further provides for a process for filling a syringe with a small quantity of a thoroughly mixed medical gas mixture comprising at least a first gas and a second gas. The process comprises the steps of providing at least a first gas and a second gas, injecting the gases into a device for mixing to form a thoroughly mixed medical gas mixture, filtering the medical gas mixture, and filling a syringe with the required amount of medical gas mixture.

### BRIEF DESCRIPTION OF THE FIGURES

FIG. 1 provides a cut-away view of the system of the present invention.

FIG. 2 provides a cut-away view of the device for mixing of the present invention.

FIG. 3 provides a view of the flow path of two gases when there are no protrusions present (prior art).

FIG. 4 provides a view of the flow path of two gases through the mixing device of the present invention which includes protrusions.

### DETAILED DESCRIPTION OF THE INVENTION

The present invention provides for a system and process for filling a syringe with a small amount of a medical gas mixture that is thoroughly mixed. As used herein the phrase “thoroughly mixed” refers to a mixture in which the components of the two or more gases are intermixed throughout the mixture (relatively equally interspersed or distributed throughout the mixture based on the ratio of the gases to one another).

The system utilized for preparing the medical gas mixture is comprised of two or more sources for gas; a device for mixing; a means for connecting the two or more sources for gas with the device for mixing; a filter system for filtering the resulting medical gas mixture, and a syringe for holding the resulting medical gas mixture. With regard to the process of the present invention, it is necessary to provide two or more gases to form the medical gas mixture. When two gases are utilized, they will be referred to herein as the first gas and the second gas. When more than two gases are referred to, they will be referred to in consecutive order (e.g., the first gas, the second gas, the third gas, the fourth gas, etc. . . . ). For purpose of the present invention, a mixture containing two gases will be utilized as the example. However, this representation is in no way meant to limit the number of gases that may be utilized in the present invention.

Typically, the gases utilized for filling a syringe will comprise one or more gases selected from typical medical gases, more precisely medical gases used in ophthalmic surgeries such as SF₆ and C₃F₈, further diluted with an inert gas such as air. With regard to the air utilized in the mixture, the air may be obtained from one of two sources: air from the room or medical grade air. When the air is obtained from the room, the air may simply be pulled from the open source. When the air utilized is medical grade, the air will be referred to as Medical Air USP that may be obtained from any number of companies that supply medical grade gases including Air Liquide. As used herein, the term “air” refers to a composition which contains a mixture of approximately 78% nitrogen, 20.9% oxygen, 0.9% argon, 0.038% carbon dioxide and trace amounts of any number of other gases and/or contaminants commonly found in the atmosphere. As used herein, the phrase “Medical Air USP” refers to a processed air which contains a mixture of 79% nitrogen and 21% oxygen along with trace amounts of allowable contaminants. While either source can be used, the Medical Air USP has already been processed (filtered) to remove unwanted contaminants. In one preferred embodiment of the present invention, the medical gas mixture will comprise two gases with the first gas being selected from SF₆ and C₃F₈ and the second gas being air. In those embodiments where SF₆ is the first gas and the second gas is air, the SF₆ is preferably present in the mixture in an amount from about 13% to about 28%, preferably from about 15% to about 26%, and even more preferably from about 18% to about 20%. In those embodiments where C₃F₈ is the first gas and the second gas is air, the C₃F₈ is preferably present in the mixture in an amount from about 10% to about 22%, preferably from about 12% to about 20%, and even more preferably from about 15% to about 16%.

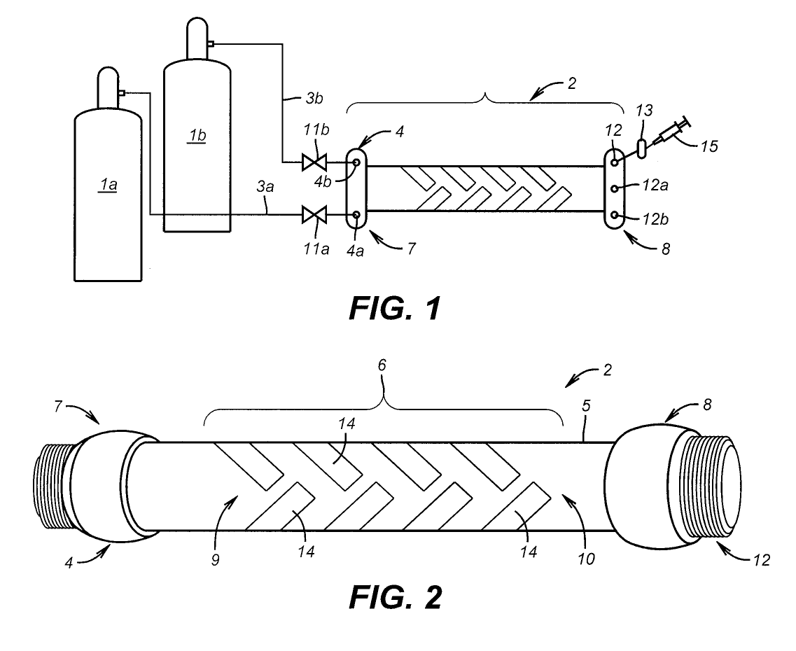

FIGS. 1 to 4 are provided herein with regard to the system and the process of the present invention. As shown in FIG. 1, which provides a view of the system of the present invention, the gases utilized in the present invention are to be supplied individually from separate gas sources (1a and 1b). These individual gas sources (by way of example, only two gas sources are utilized 1a and 1b) can be any gas source that is known in the art and may comprise bulk gas delivery systems or individual gas cylinders, including large or small composite, aluminum or steel cylinders.

As noted above, the gases to be utilized to prepare the medical gas mixtures are supplied separately to a device for mixing (2). The device for mixing (2) allows for the blending of the gases (2) of the present invention and may be any known device for the mixing or blending of gases provided that the device for mixing (2) is suitable for thoroughly mixing quantities of gases, for example, a first gas with a second gas, and allows for the withdrawal of small quantities of gas without disrupting the remaining portion of the medical gas mixture. In an additional embodiment of the present invention, the device for mixing (2) is suitable for mixing more than two gases.

In the preferred embodiment of the present invention as shown in FIG. 1 and as shown more specifically (enlarged) in FIG. 2, the device for mixing (2) comprises a housing (5) having a first end (7), a second end (8), and a mixing area (6) disposed in the housing (5) between the first end (7) and the second end (8) of the housing (5). The exterior shape of the housing (5) is not particularly critical to the present invention. However, the interior of the housing (5) is important in that it should contain a defined area that is sufficient to allow for the mixing of the two or more medical gases and the withdrawal of the resulting medical gas mixture. The interior shape of the housing (5) should be such as to not hinder the mixing of the two or more gases but rather to promote the production of a “thoroughly mixed” product as defined hereinbefore. More specifically, in order to accomplish this, the general interior shape of the mixing area (6) will be defined by an interior wall and will typically be tubular, square, round or oblong shaped, preferably tubular in shape. Reference should be made to the fact that the mixing area (6A) may be offset from the reminder of the housing (5) (off set in terms of size with the mixing area being broader in diameter than the rest of the interior portion of the housing) or the same in size with the difference being the inclusion of the protrusions (14) in the mixing are (6) (see FIG. 2). In addition, the housing (5) and the mixing area (6) should be made of a material that will not react with the gases being mixed. Such materials include, but are not limited to stainless steel, steel alloys, glass and such. In the alternative, the device can be made of any material with the inner surfaces to which the gases will come in contact being coated with a material which will not react with the gases being mixed.

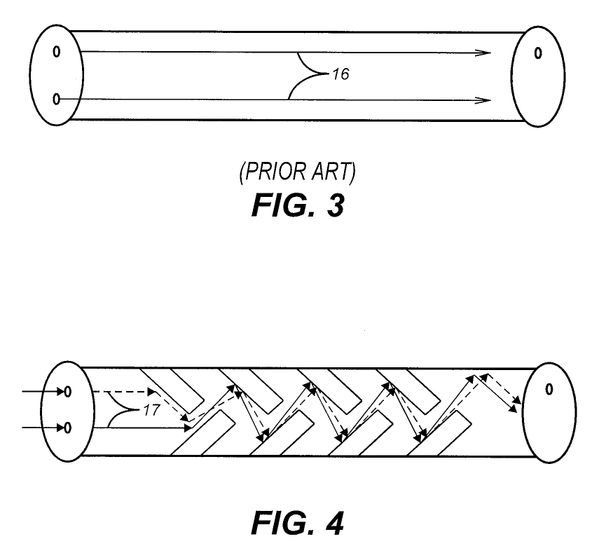

As further shown in FIG. 2, the mixing area (6) has an initial injection region (9) adjacent to the first end (7) of the housing (5) and a terminal expelling region (10) adjacent the second end (8) of the housing (5). The critical aspect with regard to the mixing area is that this area must include the presence of various protrusions, projections, vanes or fins (hereinafter collectively referred to as “protrusions”) (14) which extend from the interior wall of the mixing area of the housing (5) toward the center of the mixing area of the housing (5) in the mixing area (6). These protrusions (14) serve to break up the flow or current of any gas which is injected into the device for mixing (2). More specifically, as the various gases are injected into the device for mixing (2), the gases will be forced to travel through the channels within the mixing area (6) of the housing (5). These gases will come into contact with the protrusions (14) in the initial injection region (9) of the mixing area (6), which will cause the gas particles to bounce off the protrusions (14) and follow a path through the protrusions (14) based on these bounces and exit the mixing area (6) in the terminal expelling region (12). The protrusions (14) disrupt the path taken by the particular gas thereby creating turbulence and causing the intermingling of the gases rather than the typical relatively straight path that would be taken without the presence of the protrusions (14). The actual number of protrusions (14) present in the mixing area is not critical to the invention provided that the number present is sufficient to allow for a thorough mixing of the two or more gases. Accordingly, depending on the size of the actual device (larger devices will typically require larger numbers of protrusions in order to achieve thorough mixing, while smaller devices will typically require smaller numbers of protrusions in order to achieve thorough mixing), the mixing area could have greater than 5 protrusions (14), even more preferably greater than 15 protrusions (14) with the actual number of protrusions (14) possibly being as high as 100 or more protrusions (14) for larger devices. The arrangement of these protrusions (14) should be such as to promote the “break up” of the flow or current of any gas being injected. Accordingly, the arrangement of these protrusions (14) within the mixing area (6) in one preferred embodiment will typically be staggered (distributed on or as if on alternating sides of a center line within the mixing area; set in a zigzag row or rows; to arrange in alternating or overlapping manner). Reference is made to FIG. 3 which demonstrates the type of path taken by the gases without the presence of the protrusions (see arrow 16) and FIG. 4 which demonstrates the path taken by the gases with the presence of the protrusions (14) (see arrow 17).

The device for mixing will further contain a first manifold (4) positioned at the first end (7) of the housing (5). This first manifold will have two or more inlet ports (4a and 4b, two selected for the purpose of example only) that allow for the introduction of individual gases from the individual gas sources (1a and 1b). The inlet ports (4a and 4b) serve as the point at which the gases are injected into the device at the same time (simultaneously) thereby eliminating the need to switch out various gas cylinders when preparing a syringe (15) filled with a medical gas mixture for use in surgery such as ophthalmic surgery. The two or more inlet ports (4a and 4b) for the gases are channels that extend from the exterior of the housing (5) into the interior of the housing (5). The inlet ports (4a and 4b) allow for the securing of the connective (line) that supplies the gas to the device (2) and delivery of the gas from the gas source (1a or 1b) through the channel of the inlet port (4a or 4b) and into the mixing area (6). These inlet ports (4a and 4b) should be of the type to allow secure connection and passage of the gases into the device for mixing (2) without introducing additional contaminants or air. For example, these inlet ports may include an externally or male threaded section (not shown) that allows for connection with a corresponding female threaded of the line in order to facilitate the transport of the gas from the gas source (1a or 1b) to the device (2). The inlet port (4a or 4b) can also include any suitable sealing members, such as elastomeric O-rings (not shown), to ensure that a tight connection is established and maintained between the device and the line through which the gas is transported.

Each of the inlet ports (4a and 4b) will be attached to the corresponding means of connection (3a and 3b) which serve to connect the gas sources (1a and 1b) to the device for mixing (2). The first manifold (4) will be positioned just prior to the initial injection region (9) of the mixing area (6). This first manifold (4) will allow for the individual direct feeding of a gas into the housing (5) and flow into the initial injection region (9) of the mixing area (6) of the device for mixing (2).

The device for mixing (2) still further contains a second manifold (12) having one or more exit ports (12) for removing the resulting medical gas mixture from the mixing area (6) of the device for mixing (2). This second manifold (12) is positioned at the second end (8) of the housing (5), just after the terminal expelling region (10) of the mixing area (6). The exit port (12) is such that it allows the secure attachment of the filter systems (13) to be utilized and the syringes (15) to be filled. The exit port (12) is in the form of a channel that extends through the housing from the interior of the housing (5) to the exterior of the housing (5) of the device for mixing (2). As with the inlet ports (4a and 4b), the one or more exit ports (12) may also include an externally or male threaded section (not shown) that allows for connection with a corresponding female thread of the syringe (15) in order to facilitate the transport of the gas mixture from the mixing area (6) to the syringe (15).

As noted, the system further includes a means of connection (3a and 3b) that allows for the safe passage of the gas from the gas source (1a or 1b) to the device for mixing (2). While any type of means of connection known in the art may be utilized, the gases are typically supplied to the device for mixing (2) separately via one or more means for connection (3a and 3b) that are connected to the device for mixing (2) via the two or more inlet ports. More specifically, the gases are each supplied using one or more lines that are in the form of hoses or pipes connected at one end to the gas cylinder or vessel via a regulator (not shown) and at the other end to an inlet port (4a or 4b) for the device for mixing gases (2). As used herein the term “hoses” is generally considered to include any structure that is capable of allowing for the transfer of a gas from one point to another and includes not only hoses but also pipes, cylinders and tubes. The hoses may be of the type that are commonly used in the art of dispensing gases including, but not limited to, plastic hoses, aluminum hoses, copper hoses and stainless steel hoses. Preferably, due to the nature of the gases utilized, the hoses will be plastic hoses.

Each of the means of connection (3a and 3b) to the two or more inlets, has a corresponding proportional valve (11a and 11b) associated with it which regulates the feed of the corresponding gas from the gas source (1a or 1b) along the means of connection (3c or 3b) (lines) and into the mixing area (6) of the device for mixing (2). These proportioned valves (11a and 11b) will be positioned on the means for connection (3a and 3b) between the gas source (1a and 1b) and the corresponding inlet ports (4a and 4b). Accordingly, these proportional valves allow for the delivery of the precise amount of the first gas, the second gas or any other additional gas that may be used through the corresponding line and into the mixing area (6) of the device for mixing (2). Such proportional valves (11a and 11b) are readily known to those of ordinary skill in the art.

In some embodiments of the present invention, the means for connecting (3a or 3b) also comprises a volume flow sensor (not shown) to measure the flow of the first gas, the second gas and any additional gases utilized as they are entering the mixing area (6). In addition, an analyzer (not shown) may be associated with the exit ports to analyze the final composition of the resulting gas mixture as it is leaving the device for mixing. Devices that aid in mixing) are known in the art and include portable blending devices such as those used to dilute samples for gas chromatography (GC). Devices for mixing (2), such as those that can be utilized in the present invention, are commercially available through a variety of commercial sources including Horiba, Wasson ECE Instrumentation and Environics Gas Blends, as well as others. Such devices for mixing (2) of gases are known by a variety of names which include, but are not limited to, static microstructure mixers (including V mixers), dynamic mixers or portable mixing devices, and jetmixers.

As noted previously, the gas cylinders/vessels (gas sources) are connected to the device for mixing via a connection or line that runs from the gas cylinders/vessels to the device for mixing. When it is time to mix the two or more gases, in one embodiment, the proportional valve (11a) for the first gas will be activated to draw the correct proportion of the first gas into the device from mixing. Once this amount has been injected into the device for mixing, the valve will close. The withdrawal and subsequent injection of the second gas is simultaneously activated by opening the corresponding proportional valve (11b) to achieve the correct proportions in the resulting gas mixture. After the second gas is injected into the mixing area (6), the corresponding proportional valve (11b) is closed. The actual injection of the two gases into the device for mixing results in the mixing of the gases. The device for mixing (2) can also be agitated in order to further insure thorough mixing.

Once the two gases have been thoroughly mixed to form the resulting medical gas mixture, the gas mixture is passed from the device for mixing (2) to an optional filtering system (13) where the gas mixture is filtered to remove any contaminants that may be present, including particulate matter. The filtering system (13) utilized will include one or more filters (not shown) that allow for the removal of contaminants. For example, in one embodiment, two or more filters (preferable two to four filters) having different pore sizes may be used with the gas mixture first being passed through the filter having the larger pore size and then being passed through the filter having the smaller pore size. The filters can range in pore size from about 45 μm in size to about 0.1 μm in. In one preferred embodiment, one filter with a pore size of about 0.22 μm is used to filter the gas mixture.

While any type of filtering system (13) may be utilized, an effective and inexpensive means of filtering the resulting gas mixture is a syringe filtering system (13 plus 15) made of polypropylene housing having a filtering system inlet for gas on one side of the filter housing, a filtering system outlet for gas on the opposing side of the filter housing and a syringe allocated to the outlet. The filter housing has any number of different types of membranes encased within the housing and disposed between the filtering system inlet for gas and the filtering system outlet for gas, the membranes being selected from cellulose acetate membranes, nylon membranes, polyethersulfone membrane or Teflon PTFE membranes and the like. Such a system would allow direct connection of one end of the filter housing to the outlet of the device for mixing (2). Accordingly, as the resulting gas mixture is expelled or withdrawn from the device for mixing (2), it will automatically pass into the opening (inlet) of the filtering system (13), through the membrane where it will leave behind anything that is greater in diameter than the pore size of the membrane and out the exit (outlet) of the filtering system (13). Care should be taken when the gas mixture is expelled or withdrawn from the device for mixing since if the pressure is too strong, the membrane of the filtering system will break thereby resulting in a failure of the filtering system.

The use of a syringe filter system (13 plus 15) also allows for ease in carrying out the final step of the process—the filling of the actual syringe (15) to be used with the gas mixture. In one alternative embodiment of the present invention, it will not even be necessary for the device for mixing (2) the gas mixture to be able to expel the resulting gas mixture since in this embodiment, the full amount of gas is simply withdrawn through the filter system (13) utilizing a syringe (15) that is attached to the opposing side of the syringe filter system and into the syringe that is to hold the gas mixture until needed. In a preferred embodiment, the gas mixture is a medical gas mixture to be used in ophthalmic surgery and the syringe (15) will hold the medical gas mixture until the medical gas mixture is used in surgery. In a still further embodiment, the device for mixing (2) will be able to expel the gas mixture from the device for mixing (2) and into the filter system (13) where it will then pass through the filter system (13) and displace the stopper of the syringe (5) that is attached to the opposing side of the filter system.

In a still further embodiment, filters such as those discussed above may be positioned along the connection means prior to the entry of the gas into the device instead of, or in addition to, the filtering system discussed above.

The system that will be used for carrying out the process of the present invention includes the numerous items disclosed above. In a preferred embodiment, the system includes a system for filling a syringe with a more precisely mixed medical gas mixture comprising a source of a first gas (1a) as discussed hereinbefore, a source of a second gas (1b) as disclosed hereinbefore, a device for mixing (2) the gases as disclosed hereinbefore, a filter system (13) as disclosed hereinbefore and a medical syringe (15). The medical syringe utilized may be any type of medical syringe including plastic or glass syringes. The syringe must be such that it can hold from 1 to 20, preferably from 1 to 10 ml of gas and must be sterile. Note that syringes such as those to be used are typically available in 1, 2, 5, 10 and 20 ml sizes.

An alternative to the present invention provides for a process for filling a syringe with a gas mixture comprising the steps of providing at least a first gas source (1a) and a second gas source (1b), providing a device for mixing (2) the gases which contains a mixing area (6), pulling a vacuum on the mixing area (6), filtering a defined quantity of the first gas and then injecting the filtered first gas into the mixing area/chamber, filtering a defined quantity of the second gas and injecting the filtered second gas into the mixing area at the same time as the first gas, allowing mixing the filtered first gas with the filtered second gas in the mixing area through the passages of the gases and optionally with agitation means to form a thoroughly mixed gas mixture, and filling a syringe with the gas mixture. Preferably in this alternative, the gas mixture is a medical gas mixture to be used in eye surgery.

In this alternative process, the syringe, means for filtering, first gas and second gas are all as described hereinbefore. The main difference in this embodiment lies in the housing (5) of the device. In the process of this embodiment, the housing that is utilized in the present invention comprises a tubular container which is capable of holding and securing small quantities of gas such as those defined hereinbefore. This housing (5) includes the mixing area (6) and a means to draw a vacuum (not shown) of the housing (5), a means to draw a particular amount of the first and second gas from their respective cylinders and through the respective filter(s), a means for agitating the combined gases and finally a means for filling the syringe with the medical gas mixture. More specifically, the means for drawing a vacuum may comprise any such means known in the art including a vacuum pump or a connection to a vacuum system. By drawing a vacuum, this helps in eliminating any contaminants that may be present in the chamber. After the vacuum is drawn, a defined quantify of a first gas is filtered and injected into the chamber. This defined quantify may be withdrawn either using a valve set for dispensing a certain quantity of gas or through the use of an electronic system which may be programmed for withdrawing specific quantities of gas. Such systems are readily known in the art and are commercially available. As the first gas is withdrawn from the cylinder, it is passed through a filter to remove any contaminants that may be present in the same manner as described hereinbefore. Once the gas passes through the filter, it is injected into the chamber. The same is the carried out with regard to the second gas. With regard to the filtering, the gases may both be filtered through the same filter or each line leading from the gas cylinder to the chamber may include an individual filter. Once the gases are injected into the chamber, they are allowed to mix by virtue of this injection over the protrusions and optionally agitated using any means known in the art until a thoroughly mixed medical gas mixture is achieved. Once the mixing has been completed, the gas is injected into the syringe either by pulling back on the syringe stopper until the correct volume is dispensed into the syringe or by automated means (can be the same means (system) used to withdrew the gases from the cylinders).

While the process and apparatus of the present invention may be used in any situation where it is desirable to have a small amount of two or more gases thoroughly mixed, the process and apparatus are particularly suited for use in preparing small amounts of medical gas mixtures for intraocular surgeries.

What is claimed is:

1. A process for filing a syringe with a medical gas mixture, said process comprising the steps of:
a) providing at least a first gas and a second gas;
b) injecting the first gas into a device for mixing that comprises a hollow housing having a first end, a second end, and a mixing area disposed there between, the mixing area including multiple protrusions that extend from the interior wall of the housing throughout the mixing area toward the center of the mixing area;
c) simultaneously injecting the second gas into the device for mixing; d) allowing the first gas and the second gas to mix as the gases pass through the device for mixing device for mixing in order to form a thoroughly mixed medical gas mixture;
e) withdrawing the medical gas mixture from the device for mixing;
f) filtering the medical gas mixture; and
g) filling a syringe with the filtered medical gas mixture.

2. The process of claim 1, wherein the first gas is selected from SF₆ and C₃F₈.

3. The process of claim 2, wherein the second gas is air.

4. The process of claim 1, wherein the first gas is SF₆ and the second gas is air.

5. The process of claim 1, wherein the first gas is C₃F₈ and the second gas is air.

6. The process of claim 1, wherein the device for mixing is selected from a static microstructure mixer and a dynamic blender.

7. The process of claim 1, wherein the medical gas mixture is filtered using a membrane selected from cellulose acetate membranes, nylon membranes, polyethersulfone membranes, Teflon PTFE membranes and the like.

8. The process of claim 7, wherein the medical gas mixture is filtered using a syringe filter system having a membrane with a pore size of 0.2 μm.

9. A method for an ophthalmic treatment, wherein a gas mixture is injected by means of a syringe into an eye of a patient, wherein said gas mixture is filled into the syringe according to the process for filling of claim 1.

10. A method according to claim 9, wherein the intraocular treatment is chosen among pneumatic retinopexy, vitreous surgery, corneal surgery, ocular surgery.

11. A process for filling a syringe with a medical gas mixture, said process comprising the steps of providing at least a first gas and a second gas, providing a chamber for mixing gases, pulling a vacuum on the chamber for mixing, simultaneously filtering a defined quantity of the first gas and then injecting the filtered first gas into the chamber for mixing, filtering a defined quantity of the second gas and then injecting the filtered second gas into the chamber for mixing, mixing the filtered first gas with the filtered second gas in the chamber for mixing using agitation means to form a thoroughly mixed medical gas mixture, and filling a syringe with the medical gas mixture.

12. An system for preparing medical gas mixtures, said system comprising:
a. two or more sources for gas;
b. a device for mixing that comprises:
(i) a hollow housing having a first end, a second end, and a mixing area disposed there between, the mixing area comprising an initial injection region a terminal expelling region, and multiple protrusions that extend from the interior wall of the mixing area of the housing;
(ii) a first manifold having two or more inlet ports for the gases positioned at the first end of the housing, just prior to the initial injection region of the mixing area, the first manifold allowing for the individual direct feeding of a gas into the housing and initial injection region of the mixing area of the device, each of the two or more inlet ports having corresponding proportional valves which regulate the feed of the corresponding gas from the gas source along the corresponding means of connection and into the mixing area of the device for mixing, and
(iii) a second manifold having one or more exit ports for removing the resulting medical gas mixture from the mixing area of the device, the exit port having positioned at the second end of the housing, just after the terminal expelling region of the mixing area,
c. a means for connecting the two or more sources for gas with the device for mixing,
d. a filter system for filtering the resulting medical gas mixture, and
e. a syringe for holding the resulting medical gas mixture.

13. The system of claim 12, wherein the protrusions are arranged in a staggered fashion in order to create turbulence and allow for the break up of the flow of the gases injected.

14. The system of claim 12, wherein the means for connecting the two or more sources for gas with the device for mixing are selected from plastic hoses, copper hoses, aluminum hoses and stainless steel hoses.

15. The system of claim 14, wherein the system further comprises a proportional valve on each of the means for connecting.

16. The system of claim 15, wherein the system further comprise a volume flow sensor on each of the means for connecting.

17. The system of claim 12, wherein the device for mixing comprise a static microstructure mix or a microstructure dynamic mixture.

18. The system of claim 12, wherein the filtering system includes from one to four membranes for filtering, each of the membranes independently ranging in pore size from 45 mm to 0.1 mm.

19. The system of claim 18, wherein when two or more membranes are present, the membranes are arranged to allow for the gas mixture to pass through the membranes from largest pore size to smallest pore size.

20. The system of claim 18, wherein the membranes are selected from cellulose acetate membranes, nylon membranes, polyethersulfone membranes, or Teflon PTFE membranes.
